(19) United States
(12) Patent Application Publication
(10) Pub. No.: US 2023/0094753 A1
(43) Pub. Date: Mar. 30, 2023

(54) NANOSTRUCTURE LIPID CARRIER DELIVERY SYSTEM, COMPOSITION, AND METHODS

(71) Applicant: CAPCIUM INC., Saint-Laurent (CA)

(72) Inventors: Domenico FUOCO, Montréal (CA); Jonathan COHEN, Dollard-Des-Ormeaux (CA); Leana ROSANELLI, Montréal (CA); Keegan MCKIBBON, Montréal (CA)

(73) Assignee: CAPCIUM INC., Saint-Laurent, QC (CA)

(21) Appl. No.: 17/802,698

(22) PCT Filed: Feb. 25, 2021
(86) PCT No.: PCT/CA2021/050232

§ 371 Date: Aug. 26, 2022

Related U.S. Application Data
(60) Provisional application No. 62/981,765, filed on Feb. 26, 2020.

(51) Int. Cl. A61K 9/51; A61K 31/05

(52) U.S. Cl. CPC A61K 9/5123; A61K 31/05; A61K 9/5146

(57) ABSTRACT

Nanostructure lipid carriers, delivery systems, methods of making nanostructured lipid carriers and delivery systems, and methods of using the same are disclosed. A composition is disclosed and comprises at least one nanostructured lipid carrier, the nanostructured lipid carrier comprising a shell comprising an emulsifier, and an inner matrix comprising a solid lipid and a liquid lipid, wherein the nanostructured lipid carrier has a diameter of 50 nm or less.

### CROSS REFERENCE TO RELATED APPLICATION

This application claims the benefit of U.S. provisional patent application No. 62/981,765, which is titled DELIVERY SYSTEM FOR SOFT GEL APPLICATION, was filed Feb. 26, 2020, and is incorporated herein as if fully set forth.

### FIELD OF INVENTION

This disclosure relates to nanostructured lipid carriers including an active pharmaceutical ingredient, and delivery systems comprising the nanostructured lipid carriers.

### BACKGROUND

Active pharmaceutical ingredients may be delivered in manner that diminishes their effectiveness. For example, a hydrophobic drug delivered into the aqueous environment of the gastrointestinal tract or circulatory system may precipitate or gather in hydrophobic pools, which would lower the operational concentration of the active pharmaceutical.

Emulsion structures have been contemplated as a means of delivering hydrophobic active pharmaceutical ingredients, and include micelles, inverted micelles, liposomes, niosomes, nanoemulsions, multiple emulsions, and lipid nanoparticles.

Micelles are aggregates of surfactant molecules dispersed in a liquid colloid. For micelles in aqueous solution, the hydrophilic “head” regions of surfactant molecules contact surrounding solvent, while the hydrophobic tail regions orient to the center of the aggregate. Inverted micelles have the head groups at the center with the tails extending out (water-in-oil micelle). Nanoemulsions are similar, but on a nano-scale. Micelle structures are, however, unstable and can easily be broken.

Liposomes are spherical vesicles having at least one lipid bilayer. Liposomes can be prepared by disrupting biological membranes (such as by sonication). The major types of liposomes are multilamellar vesicles (MLV, with several lamellar phase lipid bilayers), small unilamellar liposome vesicles (SUV, with one lipid bilayer), large unilamellar vesicles (LUV), and cochleate vesicles. There are also multivesicular liposomes in which one vesicle contains one or more smaller vesicles. Liposomes are differentiated from micelles and inverted micelles in that the liposomes contain a bilayer lipid, while micelles and inverted micelles contain a monolayer lipid. Liposomes are often composed of phospholipids, especially phosphatidylcholine, but may also include other lipids, such as egg phosphatidylethanolamine, that are compatible with lipid bilayer structure. Liposomes function best in a water environment.

Niosomes are non-ionic surfactant-based vesicles. Niosomes are formed mainly by non-ionic surfactant and cholesterol incorporation as an excipient. Other excipients can also be used. Hydrophilic head groups of the inner layer orient to the center of the vesicles to form an aqueous interior. The hydrophobic tails of each layer in the bilayer orient toward each other, and the hydrophobic head groups of the outer layer interact with bulk solvent. Niosomes can entrap hydrophilic agents in the interior of the vesicles, and lipophilic agents in the vesicular membrane. These structures are more stable than micelles, but function best in an oil environment.

Nanoemulsions include a surfactant monolayer surrounding a liquid lipid core.

Multiple emulsions are complex systems where both oil in water and water in oil emulsion exists simultaneously, and are stabilized by surfactants. There are water-in-oil-in-water (w/o/w) and oil-in-water-in-oil (o/w/o) type emulsions. W/o/w emulsions are much more widely used. Multiple emulsions, like micelles, are unstable and can be easily broken.

Incorporated agents can easily move from inside to bulk medium for micelles, inverted micelles, liposomes, niosomes, nanoemulsions, multiple emulsions. In contrast, lipid nanoparticles may protect and retain incorporated agents longer than the former.

Lipid nanoparticles include solid lipid nanoparticles (SLP) and nanostructured lipid carriers (NLC). Both are nanoparticles, and both can be used to encapsulate and deliver agents. Lipid nanoparticles may also increase bioavailability of lipophilic drugs.

Solid lipid nanoparticles include solid lipid only. They have a rigid, crystalline interior encased in a solid lipid shell. There may be no structural difference between the crystalline interior and the shell other than the shell being exterior layer of lipid. Alternatively, a solid lipid nanoparticle may include different types or forms of lipids at different locations. Solid lipid nanoparticles are prone to spontaneously expelling incorporated agents at inopportune sites and times due to their intrinsic structure. For example, during long term storage, a drug incorporated in a solid lipid nanoparticle may be expelled and rendered ineffective due to its poor solution properties. As another example, nanoparticles delivered to a subject may “unload” their active pharmaceutical ingredient at the wrong site due to drug expulsion.

Nanostructured lipid nanoparticle carriers include a solid outer layer of lipid encasing a less structured inner lipid. The inner lipid may be an oil or amorphous solid. The inner lipid tends to retain a hydrophobic active pharmaceutical ingredient more stably as compared with solid lipid nanoparticle cores.

Upon administration, an active pharmaceutical ingredient may encounter hostile solution properties. Enzymes may surround it that will inactivate it or shunt it to a biologically inert or deleterious site. An active pharmaceutical ingredient's hydrophobicity may partition it to lipid handling biochemistry and sites within a subject. Each of the above-mentioned emulsion structures may be intended to enhance bioavailability of an incorporated active pharmaceutical ingredient by shielding it from undesired biochemistry or physical environments. Emulsion structures may also be crafted to include targeting moieties that result in their delivery to a desired site. An active pharmaceutical ingredient incorporated in such an emulsion may be “unloaded” from the emulsion due to a variety of factors, including but not limited to intrinsic decay of the emulsion, intrinsic diffusion of the active pharmaceutical ingredient through the emulsion, transport of the active pharmaceutical ingredient through the emulsion, a change in physical environment promoting destruction of the emulsion, and biochemical degradation of the emulsion, which may occur at a desired location for active pharmaceutical ingredient delivery.

Nanoparticles tend to be too large on the nanometer scale and aggregate. The large size may lead to inappropriate delivery sites for an active pharmaceutical ingredient. Nanoparticles also tend incorporate hydrophobic active pharmaceutical ingredients at a low level.

Small nanoparticles, nanoparticles with high active pharmaceutical ingredient loading, and new nanoparticles facilitating efficient delivery of active pharmaceutical ingredient are needed. Also needed are systems of storing and/or delivery nanoparticles.

### SUMMARY

In an aspect, the invention relates to a composition comprising at least one nanostructured lipid carrier. The nanostructured lipid carrier comprises a shell comprising an emulsifier, and an inner matrix comprising a solid lipid and/or a liquid lipid. The inner matrix may have an amorphous structure. The nanostructured lipid carrier has a diameter of 100 nm or less. The composition optionally further comprising at least one of: at least one active pharmaceutical ingredient, at least one surfactant, and a solution medium.

In an aspect, the invention relates to a composition comprising at least one nanostructured lipid carrier. The nanostructured lipid carrier comprises a shell comprising an emulsifier shell, and an inner matrix comprising a solid lipid and a liquid lipid. The inner matrix may comprises an amorphous solid. The nanostructured lipid carrier has a diameter of 50 nm or less. The nanostructured lipid carrier may comprise an anti-aggregant. The composition may comprise a medium in which the nanostructured lipid carrier(s) are incorporated. The medium may be a soft gel. The soft gel may be in the form of a soft gel capsule. The medium may be a patch, a cream, a spray, or an orally disintegrating tablet.

In an aspect, the invention relates to a nanostructured lipid carrier. The nanostructured lipid carrier comprises an outer layer and an inner matrix enclosed within the outer layer. The outer layer comprises a solid lipid. The inner matrix comprises a liquid lipid or amorphous solid lipid. The nanostructured lipid carrier has a diameter of 50 nm or less.

In an aspect, the invention relates to a method of making a delivery system. The delivery system comprises, consists essentially of, or consists of a nanostructured lipid carrier. The method comprises heating a water phase composition to a melting temperature, heating an oil phase composition to the melting temperature, mixing the water phase composition and the oil phase composition at the melting temperature to create a mixture, and homogenizing the mixture in an aqueous medium to produce the nanostructure lipid carriers. The water phase composition comprises a surfactant and water. The oil phase composition comprises a solid lipid and a liquid lipid. The oil phase may comprise an anti-aggregant, which may be a PEG derivative. A PEG derivative may be a PEG-based emulsifier. The oil phase may comprise at least one active pharmaceutical ingredient. The melting temperature is a temperature at which the water phase composition and the oil phase composition are liquid. The solid lipid is solid below the melting temperature and the liquid lipid is liquid below the melting temperature. The method may also comprise incorporating the nanostructured lipid carriers in a medium. The medium may be a soft gel. The soft gel may be in the form of a soft gel capsule. The medium may be a patch, a cream, a spray, or an orally disintegrating tablet.

In an aspect, the invention relates to a method of making nanostructured lipid carriers. The method comprises heating a water phase composition to a melting temperature, heating an oil phase composition to the melting temperature, mixing the water phase composition and the oil phase composition at the melting temperature to create a mixture, and homogenizing the mixture in an aqueous medium to produce the nanostructure lipid carriers. The water phase composition comprises a surfactant and water. The oil phase may comprise an anti-aggregant, which may be a PEG derivative. The oil phase composition comprises a solid lipid and a liquid lipid. The oil phase may comprise an active pharmaceutical ingredient. The melting temperature is a temperature at which the water phase composition and the oil phase composition are liquid. The solid lipid is solid below the melting temperature and the liquid lipid is liquid below the melting temperature.

In an aspect, the invention relates to a method of making a delivery system comprising nanostructured lipid carriers. The method comprises at least one of filtering or drying nanostructured lipid carriers or compositions comprising nanostructured lipid carriers to produce processed nanostructured lipid carriers. The method may also include dispersing the processed nanostructured lipid carriers in an excipient to prepare dispersed nanostructure lipid carriers. The method may also comprise bulk formulating the dispersed nanostructured lipid carriers to prepare formulated nanostructured lipid carriers. The method may also comprise incorporating the processed nanostructured lipid carriers, the dispersed nanostructure lipid carriers, or the formulated nanostructured lipid carriers in a soft gel.

In an aspect, the invention relates to a method of treating a subject comprising administering a composition comprising a nanostructure lipid carrier, a product of the making a delivery system that comprises a nanostructured lipid carrier, or a product of the method of making a delivery system to a subject in need thereof.

### BRIEF DESCRIPTION OF THE DRAWINGS

The following detailed description of an embodiment(s) of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIG. 1 illustrates an initial formulation process for a method of making nanostructured lipid carriers. The method of making nanostructured lipid carriers illustrated can also be a first stage in a method of making a delivery system.

FIG. 2 illustrates a bulk processing process for a method of making nanostructured lipid carriers. The first steps, including filtering and drying, of the bulk processing process may be part of a method of making nanostructured lipid carriers. The bulk processing can also be the second stage in a method of making a delivery system.

FIG. 3 illustrates a transmission electron micrograph of nanostructured lipid carriers of formula LR.15/17-PEG1.

FIG. 4 illustrates a transmission electron micrograph of nanostructured lipid carriers of formula LR.15/17/

### DETAILED DESCRIPTION

Certain terminology is used in the following description for convenience only and is not limiting. The words “right,” “left,” “top,” and “bottom” designate directions in the drawings to which reference is made. The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

As used herein, “therapeutically effective amount” of an active pharmaceutical ingredient means an amount that elicits an intended biological effect.

A nanostructured lipid carrier can also be referred to herein as an NLC.

An active pharmaceutical ingredient can also be referred to herein as an API.

Unless otherwise indicated, a percent concentration herein is a % w/w and indicates the percent of the raw material discussed in formulation prior to nanostructure lipid carrier formation:

$$[ \frac{Weight of raw material}{Total weight of premulsion} ] \times 100 = \% w / w$$

An exception to the above % w/w is the % w/w of an active pharmaceutical ingredient. Unless otherwise indicated, the % w/w of an active pharmaceutical ingredient indicates the theoretical percent of the active pharmaceutical ingredient in a dried nanostructured lipid carrier:

$$[ \frac{Weight of API}{Weight of Total NLC} ] \times 100 = \% w / w API in NLC ,$$

where Weight of Total NLC=Weight of Solid Lipid+Weight of Liquid Lipid+Weight of Emulsifier (which may be a PEG Derivative)+Weight of API+Weight of Additional Surfactant/Emulsifier. When additional components are present, the Weight of Total NLC would include the weight of the additional components added to the aforementioned.

An embodiment includes a nanostructured lipid carrier. An embodiment includes a composition comprising at least one nanostructured lipid carrier.

The nanostructured lipid carrier may comprise a shell comprising an emulsifier shell. The emulsifier in the emulsifier shell may be a PEG derivative. The nanostructured lipid carrier may comprise an inner matrix comprising a solid lipid and/or a liquid lipid. The inner matrix may comprise an amorphous solid. The chemical composition of the nanostructured lipid carrier may be similar or homogeneous from the interior toward the exterior, but with a shell formed toward the surface because of the physical environment in which the nanostructured lipid carrier was formed or exists. In such an embodiment, an emulsifier and/or solid lipid may be found in the core, but formed into a shell on the exterior because of the physical environment in which the nanostructured lipid carrier was formed or exists. Liquid lipid in such an embodiment may be found on the surface during formation, and may remain in contact with a nanostructured lipid carrier, or be carried off into bulk solvent or otherwise removed during processing or use. The nanostructured lipid carrier may also comprise a hydrophobic active pharmaceutical ingredient. The nanostructured lipid carrier may have a diameter of 100 nm or less, 95 nm or less, 90 nm or less, 85 nm or less, 80 nm or less, 75 nm or less, 70 nm or less, 65 nm or less, 60 nm or less, 55 nm or less, 50 nm or less, 45 nm or less, 40 nm or less, 35 nm or less, 30 nm or less, 25 nm or less, 20 nm or less, 15 nm or less, 10 nm or less, or 5 nm or less. The “or less” in any of the foregoing, but for 5 nm or less, may be replaced with “down to X nm” where X is one the specific foregoing sizes and less than the size stated before “or less.”

The diameter of a nanostructured lipid carrier may have a value selected from a range having a low endpoint and a high endpoint. The low endpoint may be selected from 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, and 49 nm. The high endpoint is larger than the low endpoint and may be selected from 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, and 50 nm. The range arrived at by the selection of any one of the above listed low endpoints and any one of the above listed high endpoints, with the caveat that the selected high endpoint is larger than the selected low endpoint, is within the scope of diameter ranges contemplated. Non-limiting examples of diameter ranges include the following: 1-50 nm, 5-45 nm, and 15-25 nm. The diameter of a nanostructured lipid carrier herein may be any value within the selected range. The diameter may be 20 nm.

A composition herein may comprise a plurality of nanostructured lipid carriers. The diameter of nanostructured lipid carriers therein may be as described above, but reflect the average diameter of the plurality of nanostructured lipid carriers. A composition herein may comprise a medium in which the nanostructured lipid carrier(s) are incorporated. The medium may be a soft gel. The soft gel may be in the form of a soft gel capsule. The medium may be a patch, a cream, a spray, or an orally disintegrating tablet.

A composition herein may further comprise an active pharmaceutical ingredient in the at least one of the nanostructured lipid carriers. More than one type of active pharmaceutical ingredient may be included in a nanostructured lipid carrier. Different populations of nanostructured lipid carriers in a composition herein may contain different active pharmaceutical ingredients. The active pharmaceutical ingredient(s) may be hydrophobic, or comprise a hydrophobic compound or moiety. The active pharmaceutical ingredient(s) may comprise, consist essentially of, or consist of, without limitation, at least one selected from the group consisting of a cannabinoid, cannabidiol, bicalutamide, carvediol, lovastatin, luteolin, mitotane, oridonin quercetin, spironolactone, saquinavir, saquinavir mesylate, testosterone undecanoate, thistle oil, safflower oil, sea buck thorn oil, carrot extract, thymoquinone, vinpocetine, and zerumbone. The active pharmaceutical ingredient may be cannabidiol. The active pharmaceutical ingredient may be at least one cannabinoid. The active pharmaceutical ingredient may be any hydrophobic API with a log P≥2. A composition herein may further comprise a food product. The food product may be compatible with lipid carriers. A composition herein may further comprise a vitamin, an oil, and/or a fatty acid. The vitamin may be vitamin D, E, or A. The food product, vitamin, oil, or fatty acid may be in at least one of the nanostructured lipid carriers.

The active pharmaceutical ingredient may be at a therapeutically effective amount in the composition. When there is more than one type of active pharmaceutical ingredient, each may be at a therapeutically effective amount. The amount may be 7-15% (w/w) for the amount of an active pharmaceutical ingredient. The amount may be from a low endpoint of a range of amounts to a high endpoint of the range. The low endpoint of the range may be selected from 1, 2, 3, 4, 5, 6, 7, 8, 9, 10, 11, 12, 13, 14, 15, 16, 17, 18, 19, 20, 21, 22, 23, 24, 25, 26, 27, 28, or 29% (w/w). The high endpoint of the range is higher that the low endpoint and may be selected from 2, 3, 4, 5, 6, 7, 8, 9, 10, 11, 12, 13, 14, 15, 16, 17, 18, 19, 20, 21, 22, 23, 24, 25, 26, 27, 28, 29, or 30% (w/w).

The outer layer of a nanostructured lipid carrier may comprise a solid lipid or a combination of solid lipids. A solid lipid, or combination of solid lipids, may be selected from those lipids that will remain solid at desired temperature for use of the nanostructured lipid carrier. A solid lipid, or combination of solid lipids, may be selected from those lipids that will remain solid at desired temperature for storage of the nanostructured lipid carrier. A solid lipid, or combination of solid lipids, may be selected to be solid at a desired temperature for storage of the nanostructured lipid carrier, but lose its solid character at temperatures intended during use; for example at the body temperature encountered in the subject, or at the site within a subject. A solid lipid, or combination of solid lipids, may be selected to be solid at the body temperature of the subject it will be delivered to. The subject may be an animal. The subject may be a mammal. The subject may be human.

A solid lipid, or combination of solid lipids, may be selected from lipids that have a desired solubility for the active pharmaceutical ingredient. The desired solubility may be low solubility.

The solid lipid(s) may be insoluble in water and solid at room temperature, while the melting temperature is over 25° C., over body temperature of a subject that an NLC or NLC delivery system is intended for, or over 37° C. The subject may be an animal. The subject may be a mammal. The subject may be human. The solid lipid, or combination of solid lipids, may be selected from lauroyl macroglycerides, PEG-32 stearate, glyceryl dibehenate, hydrogenated coconut PEG-32 esters, glyceryl oleate, lauroyl macroglycerides, and tricaprin.

The liquid lipid may comprises at least one lipid selected from the group consisting of apricot kernel oil PEG-6 esters, corn oil PEG-6 ester, ethoxydiglycol, glyceryl distearate, polyglyceryl-3 dioleate, glyceryl linoleate, glyceryl oleate, glyceryl monooleate, propylene glycol monolaurate, PEG-8 caprylic/capric glycerides, polyglyceryl-3-diolate, propylene glycol caprate, propylene glycol laurate, PEG-8, caprylic/capric triglyceride, caprylic/capric triglyceride, PEG-8 carylic/capric glycerides, glyceryl caprylate/caprate, propylene glycol monocaprylate, Olea europaea (Olive) oil, and Glycine soja (Soyabean) oil.

The solid lipid may comprise one or more lipid selected from glyceryl caprylate, glycerol dibehenate, and lauroyl macroglycerides, and the liquid lipid may comprise one or more liquid lipid selected from caprylic/capric triglyceride, glyceryl monooleate, propylene glycol monolaurate, hydrogenated coconut PEG-32 esters, glyceryl monooleate, and glyceryl caprylate.

The solid lipid may be at a concentration of 0.1-1% or 1-10%.

The liquid lipid may be at a concentration of 0.1-10%, 0.1-10%, 0.1-0.5%, or 1-10%.

The solid lipid may be at a concentration of 0.1-1%. The liquid lipid may be at a concentration of 0.1-1%.

The solid lipid may be at a concentration of 0.1-1%. The liquid lipid may be at a concentration of 0.1-1%.

The solid lipid may be at a concentration of 1-4%. The liquid lipid may be at a concentration of 1-2%.

The solid lipid may be at a concentration of 1-4%. The liquid lipid may be at a concentration of 0.1-1%.

A nanostructured lipid carrier herein may comprise at least one surfactant. The at least one surfactant may be selected from non-ionic surfactants, ionic surfactants, a high HLB non-ionic surfactant, a low HLB non-ionic surfactant, polysorbitan 20, sorbitan monooleate, and PEG-35 castor oil. One surfactant in a nanostructured lipid carrier herein may serve more than one function. A surfactant may be at least one of an anti-aggregant, a co-surfactant, an nanostructured lipid carrier former, or a stabilizer.

A composition comprising a nanostructured lipid carrier herein may comprise a solution medium. The solution medium may be aqueous. The solution medium may comprise water. The solution medium may be water. The solution medium may be exterior to the nanostructured lipid carrier.

A nanostructured lipid carrier herein may comprise an anti-aggregant (emulsifier) in the outer layer, in an emulsifier shell. The anti-aggregant may be a surfactant. The anti-aggregant may be a PEG derivative. The anti-aggregant may be or comprise PEG-35 castor oil.

A formulation for a nanostructured lipid carrier herein may comprise solid lipid at 0.1-1%, liquid lipid at 0.1-0.5%, surfactant/emulsifier at 15-20% or 5-30%, and a solution medium at 60-75% or 60-90%. An active pharmaceutical ingredient in a nanostructured lipid carrier may be at 0.01-1% or 0.5-20, The solid lipid may be at least one of glyceryl caprylate or glycerol dibehenate. The liquid lipid may be at least one of glyceryl monooleate, propylene glycol monolaurate, or hydrogenated coconut PEG-32 esters. The surfactant may be polysorbitan 20. The solution medium may be water.

A nanostructured lipid carrier herein may comprise solid lipid at 0.1-1%, liquid lipid at 0.1-1%, active pharmaceutical ingredient at 0.01-0.5%, surfactant comprising a high HLB non-ionic surfactant at 15-20% and low HLB non-ionic surfactant at 15-20%, and solution medium at 60-75%. The HLB surfactant may have an HLB=10-18. The solid lipid may be glycerol dibehenate. The liquid lipid may be at least one of propylene glycol monolaurate, glyceryl monooleate, or hydrogenated coconut PEG-32 esters. The high HLB non-ionic surfactant may be polysorbitan 20. The low HLB non-ionic surfactant may be sorbitan monooleate. The solution medium may be water.

A nanostructured lipid carrier herein may comprise solid lipid at 0.1-4%, optionally 1-4%, 0.1-2%, 0.1-1% or 1-2%, liquid lipid at 0.1-2%, optionally 0.1-1% or 1-2%, active pharmaceutical ingredient at 0.01-6.5%, optionally, 5-6.5%, 0.01-1%, 0.01-0.5% or 0.5-1%, surfactant comprising an anti-aggregant at 6-9% and a high HLB nonionic surfactant at 6-9%, and solution medium at 60-90%, optionally 60-80%. The solid lipid may be lauroyl macroglycerides. The liquid lipid may be glyceryl caprylate. The anti-aggregant may be PEG-35 castor oil. The high HLB nonionic surfactant may be polysorbate 20. The solution medium may be water.

A nanostructured lipid carrier herein may comprise solid lipid at 1-4%, liquid lipid is at 0.1-1%, active pharmaceutical ingredient at 0.4-15%, surfactant comprising an anti-aggregant at 6-9% and a high HLB nonionic surfactant at 6-9%, and solution medium at 60-90%. The solid lipid may be lauroyl macroglycerides. The liquid lipid may be glyceryl caprylate. The anti-aggregant may be PEG-35 castor oil. The high HLB nonionic surfactant may be polysorbate 20. The solution medium may be water.

A composition herein, which comprises at least one nanostructured lipid carrier herein, may further comprise a soft gel. The at least one nanostructured lipid carrier may be incorporated in the soft gel.

A composition herein, which comprises at least one nanostructured lipid carrier herein, may further comprise a pharmaceutically acceptable carrier.

A composition herein, which comprises at least one nanostructured lipid carrier herein, may further comprise a pharmaceutically acceptable excipient.

An embodiment comprises a method of making a delivery system. The delivery system may comprise, consist essentially of, or consist of nanostructured lipid carriers. The nanostructured lipid carriers may be one more of those described herein.

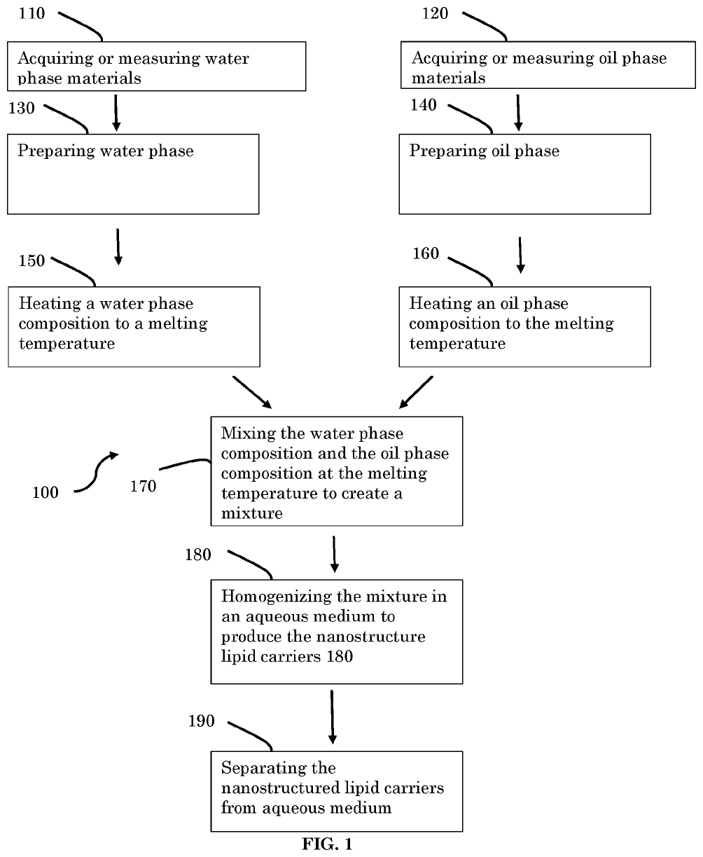

Referring to FIG. 1, an initial formulation process 100 is illustrated. The initial formulation process 100 may be common to both a method of making a delivery system, and a method of making nanostructured lipid carriers. The initial formulation process 100 represents a first stage in making a delivery system comprising making nanostructured lipid carriers. The method may comprise acquiring or measuring out starting materials for a water phase in step 110 and for an oil phase in step 120. The method may comprise preparing the water phase in step 130 and preparing the oil phase in step 140. The method comprises heating a water phase composition to a melting temperature 150, heating an oil phase composition to the melting temperature 160, mixing the water phase composition and the oil phase composition at or above the melting temperature to create a mixture 170, and homogenizing the mixture in an aqueous medium to produce the nanostructure lipid carriers 180. The homogenizing may be at a temperature below melting temperature. The homogenizing may comprise introducing the mixture to a rapidly mixing aqueous solution, wherein the rapidly mixed aqueous solution is below the melting temperature. The introducing may be dripping the mixture into the rapidly mixing aqueous solution. The difference between the melting temperature and the temperature of the homogenizing aqueous solution may be great enough that the solid lipid solidifies, or freezes, rapidly upon entry into the homogenizing aqueous solution. The solidification may be flash freezing of the solid lipid.

The water phase composition may comprise a surfactant and water. The surfactant (or emulsifier) may be a PEG derivative. The oil phase composition may comprise a solid lipid and a liquid lipid.

An oil phase and water phase may be constituted as outlined below.

| | Ingredient | w/w % |
|---|---|---|
| Oil | Solid Lipid | 1-10 |
| Phase | Liquid Lipid | 1-10 |
| | PEG-Derivative | 1-25 |
| | API | 0.5-2.0 |
| Water | Water | 5-30 |
| Phase | Surfactant | 60-90 |

An ingredient list from which solid lipid and liquid lipid may be selected for a delivery system or nanostructured lipid carrier may be as described above. An ingredient list may also be as represented in Table 1, below.

TABLE 1

| Trade Name | INCI | Company | Description |
|---|---|---|---|
| Labrafil M | Apricot Kernel | GATTEFOSSE | Oleoyl macro- |
| 1944 CS | Oil PEG 6 | | 6 glycerides/ |
| | Esters | | Oleoyl |
| | | | polyoxyl-6 |
| | | | glycerides |
| Labrafil M | Corn Oil | GATTEFOSSE | Linoleoyl |
| 2125 CS | PEG-6 Ester | | macrogol-6 |
| | | | glycerides/ |
| | | | Lineoleoyel |
| | | | polyoxyl-6 |
| | | | glycerides |
| Transcutol | Ethoxy- | GATTEFOSSE | 2-(2- |
| HP | diglycol | | Ethoxyethoxy) |
| | | | ethanol |
| Geloil SC | Glycerine | GATTEFOSSE | |
| | soja | | |
| | (Soybean) | | |
| | oil (and) | | |
| | Glyceryl | | |
| | Distearate | | |
| | (and) | | |
| | Polyglyceryl- | | |
| | 3 Dioleate | | |
| Maisine CC | Glyceryl | GATTEFOSSE | Glycerides, |
| | Linoleate | | C16-18 |
| | | | and C18- |
| | | | unsatd. |
| | | | mono-and di- |
| Peceol | Glyceryl Oleate | GATTEFOSSE | Glycerides, |
| | | | C16-18 |
| | | | and C18- |
| | | | unsatd. |
| | | | mono-and di- |
| Labrasol | PEG-8 | GATTEFOSSE | Caprylocaproly |
| ALF | Caprylic/ | | macrogol-8 |
| | Capric | | glycerides/ |
| | Glycerides | | polyoxyl-8 |
| | | | glycerides |
| Plurol | Polyglyceryl- | GATTEFOSSE | |
| Oleique | 3 diolate | | |
| CC 497 | | | |
| Capryol 90 | Propylene | GATTEFOSSE | |
| | Glycol | | |
| | Caprate | | |
| Lauroglycol | Propylene | GATTEFOSSE | |
| 90 | Glycol | | |
| | Caprate | | |
| Gelucine | PEG-32 | GATTEFOSSE | |
| 48/16 | Stearate | | |
| Pellets | | | |
| Compritol | Glyceryl | GATTEFOSSE | Glycerides, |
| 888 ATO | Dibehenate | | C16-22 |
| | | | mono-, |
| | | | di-, tri-(or |
| | | | Docosanoic |
| | | | acid, |
| | | | monesters |
| | | | with 1,2,3- |
| | | | propanetriol) |
| | | | (or 2-hydroxy- |
| | | | propane-1,3- |
| | | | diyldidocasanoate) |
| CARBO- | PEG-8 | Dow | Polyethylene |
| WAX | | | glycol |
| SENTRY | | | |
| Polyethylene | | | |
| Glycol | | | |
| 400 | | | |
| Gelucire | Hydrogenated | GATTEFOSSE | Lauroyl |
| 44/14 | Coconut | | macrogol-32 |
| | PEG-32 | | glycerides/ |
| | Esters | | Lauroyl |
| | | | polyoxyl- |
| | | | 32 glycerides |
| Capmul | Glyceryl | Abitec | Alkyl |
| MCM C8 | Caprylate | | Distribution: |
| | | | 97.3% |
| | | | caprylic |
| | | | acid & 2.7%- |
| | | | Capric |
| | | | acid |
| Capmul | Glyceryl | Abitec | Glyceryl |
| GMO-50 | Oleate | | Mono-oleate |
| Acconon | Lauroyl | Abitec | |
| C-44 | Macroglycerides | | |
| Labrafac | Caprylic/ | GATTEFOSSE | Glycerides, |
| Lipophile | Capric | | mixed |
| WL1349 | Triglyceride | | decanoyl |
| | | | and octanoyl |
| | | | (or glycerides, |
| | | | C8-10) (or |
| | | | triglycerides, |
| | | | medium chain) |
| Captex 355 | Caprylic/ | Abitec | Alkyl |
| | Capric | | Distribution: |
| | Triglyceride | | 58.5%- |
| | | | Caprylic |
| | | | Acid, 41.2%- |
| | | | Capric |
| | | | Acid & 0.2%- |
| | | | Lauric Acid |
| Acconon | PEG-8 | Abitec | PEG-8 |
| MC8-2, | Carylic/ | | caprylic/ |
| EP/NF | Capric | | capric |
| | Glycerides | | glyceride |
| Capmul | Glyceryl | Abitec | Alkyl |
| MCM | Caprylate/ | | Distribution: |
| | Caprate | | 83.1%- |
| | | | octanoic |
| | | | acid & 16.9% |
| | | | decanoic acid |
| Capmul | Propylene | Abitec | Alkyl |
| PG-8 | Glycol | | Distribution: |
| | Monocaprylate | | 99.8%- |
| | | | Caprylic |
| | | | Acid & 0.2%- |
| | | | Capric Acid |
| Olive Oil | Olea | Capcium | |
| | Europaea | | |
| | (Olive Oil) | | |
| Soybean Oil | Glycine | Capcium | |
| | Soja (Soyabean | | |
| | Oil) | | |
| β-Cyclodextrin | Hydroxypropyl | SIGMA- | |
| | Cyclodextrin | ALDRICH | |
| Captex 1000 | Tricaprin | Abitec | |

The melting temperature may be a temperature at which the water phase composition and the oil phase composition are liquid. The solid lipid may be solid below the melting temperature and the liquid lipid may liquid below the melting temperature. The liquid lipid may be liquid at the temperature of storage. The liquid lipid may be liquid at the temperature of use. The combination of solid lipid and liquid may create an amorphous solid. The melting temperature may be 66° C. The melting temperature may depend on the melting point of the solid lipid. For example, the melting point of glyceryl dibehenate is about 72° C. To allow that the solid lipid has been melted, a water bath can be above the melting point. In the case of glyceryl dibehenate, the water bath may be above 72° C. The water bath may be but is not limited to 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30° C. above the solid lipid melting point, or in a range above the solid lipid melting point between any two of the foregoing. The water bath may be 20° C. above the solid lipid melting point. For the case of glyceryl dibehenate, the water bath may be about 90° C.

Still referring to FIG. 1, mixing the water phase composition and the oil phase composition at the melting temperature to create a mixture 170 may comprise adding the oil phase composition into the water phase composition while mixing. The water and oil phases may be at the same temperature.

Also still referring to FIG. 1, homogenizing the mixture in an aqueous medium to produce the nanostructure lipid carriers 190 may comprise drop-wise addition of the mixture into the aqueous medium. Any high shear mixer or homgenizer that can reach high speeds may be utilized. Homogenizing may comprise rapid agitation of the mixture in the aqueous medium. For example, a homogenizer may be set to setting 1=4,000 rpm. Bulk could be mixed for ˜2 minutes. Once mixed bulk bubbles may be allowed to settle (˜1 hour). Speed may depend on the formula used. Some formulas do not require much mixing while others need extra to form an NLC. 4,000 rpm may be the minimum speed required. The aqueous medium may be at a temperature lower than the melting temperature. The temperature of the aqueous medium may be 2-4° C. or 1-8° C. The amount of formula added to cold water may be 15 g formula to 45 g cold water. The temperature may be maintained during homogenization by cooling. The aqueous medium may be water.

An initial formulation process herein may start at any one of the steps illustrated in FIG. 1. For example, water and oil phase compositions may be acquired pre-made and the process may start at steps 150 and 160. The method may also include separating the nanostructured lipid carriers from aqueous medium 190.

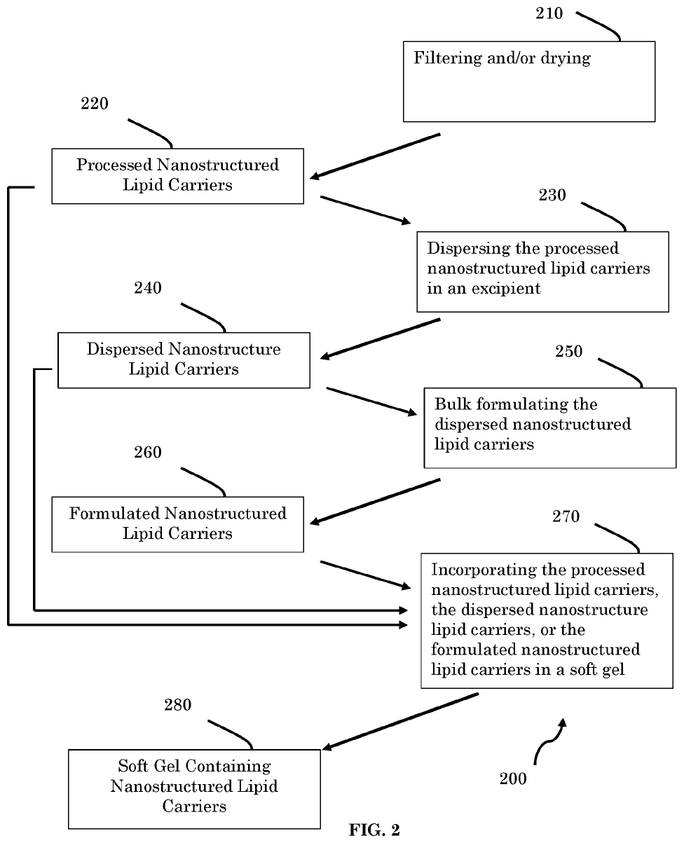

Referring to FIG. 2, a bulk processing process is illustrated. The first steps, including filtering and drying 210, of the bulk processing process may be part of a method of making nanostructured lipid carriers. The bulk processing can also be the second stage in a method of making a delivery system. The method of making a delivery system may comprise a step 210 of at least one of filtering or drying nanostructured lipid carriers obtained from an initial formulation process as described above. The filtering or drying step 210 produces processed nanostructured lipid carriers 220. The method may comprise a step 230 dispersing the nanostructured lipid carriers in an excipient to produce dispersed nanostructured lipid carriers 240. The method may comprise a step 250 of bulk nanostructured lipid carrier formulating to produce formulated nanostructured lipid carriers 260. This step may be utilized for the NLC processing into final goods (e.g., soft gel capsules, creams, etc). For gel capsules, the bulk processing may include drying of the NLC's to remove water, and then incorporating them into the gel capsule bulk for final product manufacturing. The method may also comprise a step 270 of incorporating the processed nanostructured lipid carriers, the dispersed nanostructure lipid carriers, or the formulated nanostructured lipid carriers in a soft gel to produce soft gel containing nanostructured lipid carriers 280. The method may comprise at least two of the filtering or drying 210, dispersing 230, bulk nanostructured lipid carrier formulating 250, or incorporating 270 steps.

An embodiment comprises a method of making nanostructured lipid carriers. The method comprises the heating a water phase composition to a melting temperature, heating an oil phase composition to the melting temperature. mixing the water phase composition and the oil phase composition at the melting temperature to create a mixture, and homogenizing the mixture in an aqueous medium to produce the nanostructure lipid carriers, as discussed above with respect to the method of making a delivery system.

A method of making a delivery system or a method of making a nanostructured lipid carrier herein comprise at least one of determining lipids suitable as the solid lipid or determining the lipids suitable as a liquid lipid. Determining lipids suitable as a solid lipid may comprise identifying lipids that will be solid at the temperature of use and/or storage. Determining lipids suitable as a solid lipid may comprise may comprise identifying lipids in which the active pharmaceutical ingredient has low solubility or in which the active pharmaceutical ingredient is insoluble. Determining lipids suitable as a liquid lipid may comprise identifying lipids that will remain liquid at the temperature of use and/or storage. Determining lipids suitable as a liquid lipid may comprise identifying lipids that will remain an amorphous solid at the temperature of use and/or storage. Determining lipids suitable as a liquid lipid may comprise identifying lipids in which the active pharmaceutical ingredient is soluble.

An example of determining lipids suitable is shown in Table 2, below. Approximately 0.345 g of CBD was exposed to 10 ml of the listed lipid and solubility was assessed.

TABLE 2

| SOLVENT | SOLVENT | |
|---|---|---|
| # | EXCIPIENTS TESTED | SOLUBILITY |
| 1 | Apricot Kernel Oil | Medium CBD Solubility |
| | PEG-6 Esters | |
| 2 | Corn Oil PEG-6 Ester | Medium CBD Solubility |
| 3 | Ethoxydiglycol | Stability Problematic |
| 4 | Glycerine soja (Soyean) | Medium CBD Solubility |
| | oil (and) Glyceryl | |
| | Distearate (and) | |
| | Polyglyceryl-3 Dioleate | |
| 5 | Glyceryl Linoleate | Bad CBD Solubility |
| 6 | Glyceryl Oleate | Bad CBD Solubility |
| 7 | PEG-8 Caprylic/ | Stability Problematic |
| | Capric Glycerides | |
| 8 | Polyglyceryl-3-diolate | Bad CBD Solubility |
| 9 | Propylene Glycol Caprate | Good CBD Solubility |
| 10 | Propylene Glycol Laurate | Medium CBD Solubility |
| 11 | PEG-32 Stearate | N/A-Solid Lipid |
| 12 | Glyceryl Dibehenate | N/A-Solid Lipid |
| 13 | PEG-8 | Medium CBD Solubility |
| 14 | Hydrogenated Coconut | N/A-Solid Lipid |
| | PEG-32 Esters | |
| 15 | Glyceryl Caprylate | N/A-Solid Lipid |
| 16 | Glyceryl Oleate | N/A-Solid Lipid |
| 17 | Lauroyl Macroglycerides | N/A-Solid Lipid |
| 18 | Caprylic/Capric | Medium CBD Solubility |
| | Triglyceride | |
| 19 | PEG-8 Carylic/ | Good CBD Solubility |
| | Capric Glycerides | |
| 20 | Glyceryl Caprylate/ | Medium CBD Solubility |
| | Caprate | |
| 21 | Propylene Glycol | Good CBD Solubility |
| | Monocaprylate | |
| 22 | Olea Europaea | Bad CBD Solubility |
| | (Olive) Oil | |
| 23 | Glycine Soja | Medium CBD Solubility |
| | (Soybean) Oil | |

From the results in Table 2, a suitable liquid lipid for a CBD containing NLC may be selected from those showing Good CBD solubility: propylene glycol caprate, PEG-8 caprylic/capric glycerides, and propylene glycol monocaprylate.

Stability may be assessed by any means specific to the active pharmaceutical ingredient. Stability may be assessed by a degradation study. A degradation study may be conducted by exposing the active pharmaceutical ingredient in a selected lipid, or combinations of components, and analyzing the integrity of the active pharmaceutical ingredient after a certain time of exposure. The exposure may be at a certain temperature, physical environment, or chemical environment. For example the combination may be left at room temperature or body temperature for a given amount of time. The analysis may be HPLC analysis.

Properties that may be considered in matching an active pharmaceutical ingredient with the remaining components of a nanostructured lipid carrier or system containing the same include, but are not limited to Log P.

An embodiment comprises a method of making a delivery system comprising nanostructured lipid carriers. The method may comprise filtering or drying nanostructured lipid carriers obtained by any method, or pre-made, to obtain processed nanostructured lipid carriers. The method may comprise dispersing processed nanostructured lipid carriers obtained by any method, or pre-made, to prepare dispersed nanostructure lipid carriers. The method may comprise bulk formulating dispersed nanostructured lipid carriers obtained by any method, or pre-made, to prepare formulated nanostructured lipid carriers. The method may comprise incorporating processed nanostructured lipid carriers, dispersed nanostructure lipid carriers, or the formulated nanostructured lipid carriers in a soft gel.

An embodiment comprises a method of treating a subject comprising administering any composition or nanostructured lipid carrier herein, or made by a method herein, to a subject in need thereof. The subject may be an animal. The subject may be a mammal. The subject may be a human.

Embodiments list. The following list includes particular, non-limiting embodiments contemplated, and does not exclude embodiments otherwise disclosed herein.

1. A composition comprising at least one nanostructured lipid carrier, the nanostructured lipid carrier comprising a shell comprising an emulsifier, and an inner matrix comprising a solid lipid and/or a liquid lipid, wherein the nanostructured lipid carrier has a diameter of 100 nm or less; the composition optionally further comprising at least one of: at least one active pharmaceutical ingredient, at least one surfactant, and a solution medium. The inner matrix may have an amorphous structure. The chemical composition of the nanostructured lipid carrier may be similar or homogeneous from the interior toward the exterior, but with the shell formed toward the surface because of the physical environment in which the nanostructured lipid carrier was formed or exists. In such an embodiment, an emulsifier and/or solid lipid may be found in the core, but formed into a shell on the exterior because of the physical environment in which the nanostructured lipid carrier was formed or exists. Liquid lipid in such an embodiment may be found on the surface during formation, and may remain in contact with a nanostructured lipid carrier, or be carried off into bulk solvent or otherwise removed during processing or use.

2. The composition of embodiment 1, wherein the diameter is a value selected from a range having a low endpoint and a high endpoint,

optionally where the low endpoint is selected from the group consisting of 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, and 99 nm, and the high endpoint is larger than the low endpoint and is selected from the group consisting of 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, and 100 nm, preferably where the low endpoint is 1 nm and the high endpoint is 50 nm, or the low endpoint is 5 nm and the high endpoint is 45 nm, or the low endpoint is 15 nm, and the high endpoint is 25 nm, or preferably where the diameter is 20 nm.

3. The composition of embodiment 1 or 2, wherein the at least one nanostructured lipid carrier comprises a plurality of nanostructured lipid carriers, and the diameter is the average diameter of the plurality of nanostructured lipid carriers.

4. The composition of any one or more of embodiments 1-3, wherein the composition comprises the at least one active pharmaceutical ingredient in at least one, preferably most and more preferably all, of the nanostructured lipid carriers, optionally where the at least one active pharmaceutical ingredient is hydrophobic, optionally where the at least one active pharmaceutical ingredient is selected from the group consisting of a cannabinoid, cannabidiol, bicalutamide, carvediol, lovastatin, luteolin, mitotane, oridonin quercetin, spironolactone, saquinavir, saquinavir mesylate, testosterone undecanoate, thistle oil, safflower oil, sea buck thorn oil, carrot extract, thymoquinone, vinpocetine, and zerumbone;

and/or wherein the composition comprises a food product, optionally where the food product is compatible with lipid carriers; and/or wherein composition comprises a vitamin, an oil, and/or a fatty acid, optionally where the vitamin is vitamin D, E, or A; and/or wherein the food product, vitamin, oil, or fatty acid may be in at least one of the nanostructured lipid carriers.

5. The composition of embodiment 4, wherein the active pharmaceutical ingredient comprises, consists essentially of, or consists of cannabidiol.

6. The composition of embodiment 4 or 5, wherein substantially all of the nanostructured lipid carriers comprise the at least one active pharmaceutical ingredient

7. The composition of any one or more of embodiments 1-6, wherein the emulsifier is a PEG derivative.

8. The composition of any one or more of embodiments 1-7, wherein the solid lipid is at least one selected from the group consisting of lauroyl macroglycerides, PEG-32 stearate, glyceryl dibehenate, hydrogenated coconut PEG-32 esters, glyceryl oleate, lauroyl macroglycerides, and tricaprin.

9. The composition of any one or more of embodiments 1-8, wherein the liquid lipid is selected from the group consisting of apricot kernel oil PEG-6 esters, corn oil PEG-6 ester, ethoxydiglycol, glyceryl distearate, polyglyceryl-3 dioleate, glyceryl linoleate, glyceryl oleate, glyceryl monooleate, propylene glycol monolaurate, PEG-8 caprylic/capric glycerides, polyglyceryl-3-diolate, propylene glycol caprate, propylene glycol laurate, PEG-8, caprylic/capric triglyceride, caprylic/capric triglyceride, PEG-8 carylic/capric glycerides, glyceryl caprylate/caprate, propylene glycol monocaprylate, Olea europaea (Olive) oil, and Glycine soja (Soyabean) oil.

10. The composition of any one or more of embodiments 1-9, wherein the solid lipid comprises a lipid selected from glyceryl caprylate, glycerol dibehenate, and lauroyl macroglycerides, and the inner matrix comprises a liquid lipid selected from caprylic/capric triglyceride, glyceryl monooleate, propylene glycol monolaurate, hydrogenated coconut PEG-32 esters, glyceryl monooleate, and glyceryl caprylate.

11. The composition of any one or more of embodiments 1-10, wherein the solid lipid is at a concentration of 0.1-1% and the liquid lipid is at a concentration of 0.1-0.5%, or the solid lipid is at a concentration of 0.1-1% and the liquid lipid is 0.1-1%, or the solid lipid is at a concentration of 0.1-1% and the liquid lipid is at a concentration of 0.1-1%, or the solid lipid is at a concentration of 1-4% and the liquid lipid is at a concentration of 1-2%, or the solid lipid is at a concentration of 1-4% and the liquid lipid is at a concentration of 0.1-1%.

12. The composition of any one or more of embodiments 1-11 comprising the at least one surfactant, optionally where the at least one surfactant is selected from non-ionic surfactants, ionic surfactants, a high HLB non-ionic surfactant, a low HLB non-ionic surfactant, polysorbitan 20, sorbitan monooleate, PEG-35 castor oil.

13. The composition of any one or more of embodiments 1-12 comprising the solution medium, optionally where the solution medium is water.

14. The composition of embodiment 12 or 13, wherein the at least one surfactant is an anti-aggregant, optionally where the anti-aggregant comprises PEG-35 castor oil.

15. The composition of any one or more of embodiments 1-14, wherein the solid lipid is at 0.1-1%, the liquid lipid is at 0.1-0.5%, the active pharmaceutical ingredient is at 0.01-1%, surfactant is at 15-20%, and the solution medium is at 60-75%.

16. The composition of any one or more of embodiments 1-15, wherein the solid lipid is glyceryl caprylate or glycerol dibehenate, the liquid lipid is glyceryl monooleate, propylene glycol monolaurate, or hydrogenated coconut PEG-32 Esters, the surfactant is polysorbitan 20, and the solution medium is water.

17. The composition of any one or more of embodiments 1-14, wherein the solid lipid is at 0.1-1%, the liquid lipid is at 0.1-1%, the active pharmaceutical ingredient is at 0.01-0.5%, the surfactant comprises a high HLB non-ionic surfactant at 15-20% and low HLB non-ionic surfactant at 15-20%, and the solution medium is at 60-75%.

18. The composition of any one or more of embodiments 1-14 or 17, wherein the solid lipid is glycerol dibehenate, the liquid lipid is propylene glycol monolaurate, glyceryl monooleate, or hydrogenated coconut PEG-32 esters, the high HLB non-ionic surfactant is polysorbitan 20, the low HLB non-ionic surfactant is sorbitan monooleate, and the solution medium is water.

19. The composition of any one or more of embodiments 1-14, wherein the solid lipid is at 0.1-4%, optionally 1-4%, 0.1-2%, 0.1-1% or 1-2%, the liquid lipid is at 0.1-2%, optionally 0.1-1% or 1-2%, the active pharmaceutical ingredient is at 0.01-6.5%, optionally, 5-6.5%, 0.01-1%, 0.01-0.5% or 0.5-1%, the surfactant comprises an anti-aggregant at 6-9% and a high HLB nonionic surfactant at 6-9%, and the solution medium is at 60-90%, optionally 60-80%.

20. The composition of any one or more of embodiments 1-14 or 19, wherein the solid lipid is lauroyl macroglycerides, the liquid lipid is glyceryl caprylate, anti-aggregant is PEG-35 castor oil, the high HLB nonionic surfactant is polysorbate 20, and the solution medium is water.

21. The composition of any one or more of embodiments 1-14, wherein the solid lipid is at 1-4%, the liquid lipid is at 0.1-1%, the active pharmaceutical ingredient is at 0.4-15%, the surfactant comprises an anti-aggregant at 6-9% and a high HLB nonionic surfactant at 6-9%, and the solution medium is at 60-90%.

22. The composition of any one or more of embodiments 1-14 or 21, wherein the solid lipid is lauroyl macroglycerides, the liquid lipid is glyceryl caprylate, anti-aggregant is PEG-35 castor oil, the high HLB nonionic surfactant is polysorbate 20, and the solution medium is water.

23. The composition of any one or more of embodiments 1-22, wherein the at least one active pharmaceutical ingredient comprises cannabidiol, optionally wherein the cannabidiol is at a concentration of 7-15%.

24. The composition of any one or more of embodiments 1-23, wherein the at least one active pharmaceutical ingredient is at a concentration of 7-15%.

25. The composition of any one or more of embodiments 1-24 further comprising a soft gel, wherein the at least one nanostructured lipid carrier is incorporated in the soft gel.

26. A method of making nanostructured lipid carriers, the method comprising: heating a water phase composition to at least a melting temperature; heating an oil phase composition to at least the melting temperature; mixing the water phase composition and the oil phase composition at least at the melting temperature to create a mixture; and homogenizing the mixture in an aqueous medium to produce the nanostructured lipid carriers, wherein the water phase composition comprises an emulsifier and water, the oil phase composition comprises a solid lipid and a liquid lipid, and the melting temperature is a temperature at which the solid lipid melts, the water phase composition and the oil phase composition are liquid at or above the melting temperature, and the solid lipid is solid below the melting temperature and the liquid lipid is liquid below the melting temperature.

27. The method of embodiment 26, wherein the mixing comprises adding the oil phase composition into the water phase composition while mixing.

28. The method of embodiment 26 or 27, wherein the homogenizing comprises drop-wise addition of the mixture into the aqueous medium.

29. The method any one or more of embodiments 26-28, wherein the homogenizing comprises rapid agitation of the mixture in the aqueous medium.

30. The method any one or more of embodiments 26-29, wherein the aqueous medium is at a temperature lower than the melting temperature.

31. The method any one or more of embodiments 26-30, wherein the temperature is 2-4° C. or 1-8° C.

32. The method any one or more of embodiments 26-31, wherein the aqueous medium is water.

33. The method any one or more of embodiments 26-32, wherein the melting temperature is 20° above the solid lipid melting temperature, or 66° C., or 90° C.

34. The method any one or more of embodiments 26-33 further comprising at least one of filtering or drying the nanostructured lipid carriers obtained from the homogenizing.

35. A method of making a delivery system comprising dispersing the nanostructured lipid carriers of any one or more of embodiments 1-25 or made by the method of any one or more of embodiments 26-34 in an excipient.

36. The method embodiment 35 further comprising bulk nanostructured lipid carrier formulating.

37. A method comprising any one or more of embodiments 26-36 and further comprising incorporating the nanostructured lipid carriers in a soft gel.

38. A method comprising any one or more of embodiments 26-37, wherein the emulsifier is a PEG derivative.

39. The method any one or more of embodiments 26-38, wherein the liquid lipid remains liquid at one of 25° C. or 37° C.

40. The method any one or more of embodiments 26-39, wherein the emulsifier is a PEG derivative.

41. A method of making a delivery system comprising nanostructured lipid carriers, the method comprising: at least one of filtering or drying the composition of any one or more of embodiments 1-25 or the nanostructured lipid carriers prepared by the method of one or more of embodiments 26-34 or 39-40 to prepare processed nanostructured lipid carriers.

42. The method of embodiment 41 further comprising dispersing the processed nanostructured lipid carriers in an excipient to prepare dispersed nanostructure lipid carriers.

43. The method of embodiment 42 further comprising bulk formulating the dispersed nanostructured lipid carriers to prepare formulated nanostructured lipid carriers.

44. The method of any one or more of embodiments 41-43 further comprising incorporating the processed nanostructured lipid carriers, dispersed nanostructure lipid carriers, or the formulated nanostructured lipid carriers in a soft gel.

45. A method of treating a subject comprising administering the composition of any one of embodiments 1-25 or a product of any one or more of embodiments 26-44 to a subject in need thereof.

### EXAMPLES

The following examples illustrate particular embodiments herein, but are not limiting to the scope of the remaining embodiments described throughout the specification.

As used herein, primary lipid means solid lipid and secondary lipid means liquid lipid.

### Example 1

Formula LR.15/17-PEG1 was created. This formula for nanostructured lipid carriers included Lauroyl Macroglycerides (primary lipid) at 0.1-1%, Glyceryl Caprylate (secondary lipid) at 0.1-1%, PEG-35 Castor Oil (co-surfactant, anti-aggregation of nanoparticles) at 6-9%, API (Active Pharmaceutical Ingredient) at 0.01-0.5%, water (solution medium, NLC former) at 60-90%, and Polysorbitan 20 (High HLB non-ionic surfactant, NLC former and stabilizer) at 6-9%. The API was CBD. Formula LR.15/17-PEG1 is shown below in Table 3.

TABLE 3

| Ingredient | % w/w |
|---|---|
| Lauroyl Macroglycerides | 0.1-1 |
| Glyceryl Caprylate | 0.1-0.5 |
| PEG-35 Castor Oil | 6-9 |
| Cannabidiol | 0.01-1 |
| Water | 60-90 |
| Polysorbate 20 | 6-9 |

This formula was tested in DLS, Transmission Electron Microscopy (TEM) and Scattering Electron Microscopy (SEM) for nanoparticle size. This formula was found to have the smallest size and most homogeneity among the nanoparticles. It was decided that the concentration of API was needed to be doubled in the formula.

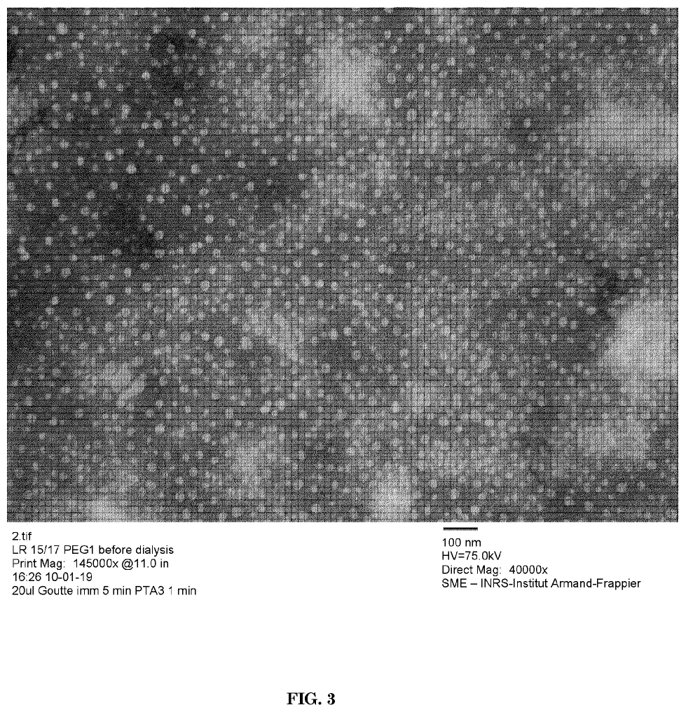

Results: Formula was tested in DLS, Transmission Electron Microscopy (TEM) and Scattering Electron Microscopy (SEM) for nanoparticle size. This formula was found to have an average nanoparticle size (diameter) of 24.93 nm, and high homogeneity. FIG. 3 illustrates a the TEM photo of the NLC Formula of this example (LR.15/17-PEG1).

### Example 2

Formula LR.15/17 was created. This formula for nanostructured lipid carriers included Lauroyl Macroglycerides (primary lipid) at 0.1-1%, Glyceryl Caprylate (secondary lipid) at 0.1-1%, PEG-35 Castor Oil (co-surfactant, anti-aggregation of nanoparticles) at 6-9%, API (Active Pharmaceutical Ingredient) at 0.5-1%, water (solution medium, NLC former) at 60-90%, and Polysorbitan 20 (High HLB non-ionic surfactant, NLC former and stabilizer) at 6-9%. Formula LR.15/17 is shown below in Table 4.

TABLE 4

| Ingredient | % w/w |
|---|---|
| Lauroyl Macroglycerides | 1-2 |
| Glyceryl Caprylate | 1-2 |
| PEG-35 Castor Oil | 6-9 |
| Cannabidiol | 0.5-1 |
| Water | 60-80 |
| Polysorbate 20 | 6-9 |

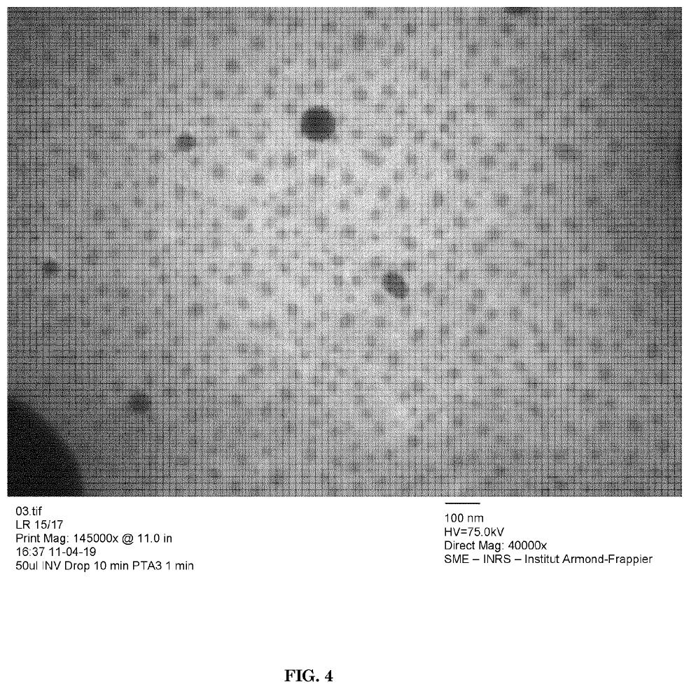

Formula LR.15/17 was tested in TEM for nanoparticle size. It was determined that the size remained the same as for Formula LR.15/17-PEG1. FIG. 4 is the TEM photo for LR.15/17 nanostructured lipid carriers.

### Example 3

Increased dosage of API was achieved with liquid lipid that had high affinity for CBD (deduced from solubility test, see Table 2, above)

### Example 4

Formula LR.17/9 was created. This formula for nanostructured lipid carriers included Lauroyl Macroglycerides (primary lipid) at 1-4%, Propylene Glycol Caprate (secondary lipid, high solubility of API) at 1-2%, PEG-35 Castor Oil (co-surfactant, anti-aggregation of nanoparticles) at 6-9%, API (Active Pharmaceutical Ingredient) at 0.5-1%, water (solution medium, NLC former) at 60-90%, Polysorbitan 20 (High HLB non-ionic surfactant, NLC former and stabilizer) at 6-9%. Formula LR.17/9 is shown below in Table 5.

TABLE 5

| Ingredient | % w/w |
|---|---|
| Lauroyl Macroglycerides | 1-4 |
| Glyceryl Caprylate | 1-2 |
| PEG-35 Castor Oil | 6-9 |
| Cannabidiol | 0.5-1 |
| Water | 60-80 |
| Polysorbate 20 | 6-9 |

Visual inspection of a formula being completely transparent leads to a deduction of nanoparticle size <100 nm. Formula LR.17/9 was visually examined and it was determined that the nanoparticle size is similar to the previous formula (LR.15/17), due to colorless and transparent solution.

### Example 5

Further increases in API were experimented with. Formula LR.17/9-A20 was created. This formula for nanostructured lipid carriers included Lauroyl Macroglycerides (primary lipid) at 1-4%, Propylene Glycol Caprate (secondary lipid, high solubility of API) at 0.1-1%, PEG-35 Castor Oil (co-surfactant, anti-aggregation of nanoparticles) at 6-9%, API (Active Pharmaceutical Ingredient) at 5-6.5%, water (solution medium, NLC former) at 60-90%, Polysorbitan 20 (High HLB non-ionic surfactant, NLC former and stabilizer) at 6-9%. Formula LR.17/9 is shown below in Table 6.

TABLE 6

| Ingredient | % w/w |
|---|---|
| Lauroyl Macroglycerides | 1-4 |
| Glyceryl Caprylate | 0.1-1 |
| PEG-35 Castor Oil | 6-9 |
| Cannabidiol | 5-6.5 |
| Water | 60-80 |
| Polysorbate 20 | 6-9 |

Nanostructured lipid carriers of formula LR.17/9-A20 were determined to have an increased size due to visual indication of opaque finished formula.

### Example 6

Formula LR.17/9, which gave nanoparticles of ˜40 nm was used to incrementally increase the API and determine loading capacity of the nanoparticles so made.

Formulas testing different % of API were as follows: Lauroyl Macroglycerides (primary lipid) at 1-4%, Propylene Glycol Caprate (secondary lipid, high solubility of API) at 0.1-1%, PEG-35 Castor Oil (co-surfactant, anti-aggregation of nanoparticles) at 6-9%, API (Active Pharmaceutical Ingredient (CBD)) at varying %, water (solution medium, NLC former) at 60-90%, Polysorbitan 20 (High HLB non-ionic surfactant, NLC former and stabilizer) at 6-9%. Table 7, below, shows the API ranges as they were incrementally increased in the base formula LR.17/9 in sub-samples A0.5, A1, A1.5, and A-2.

TABLE 7

| | API % w/w range in | API % range in |
|---|---|---|
| Code | Formula (Cannabidiol) | Nanoparticle |
| LR.17/9-A0.5 | 0.4-0.6 | 2.2-2.4 |
| LR.17/9-A1 | 0.9-1.1 | 4.2-4.4 |
| LR.17/9-A1.5 | 1.4-1.6 | 6.3-6.5 |
| LR.17/9-A2 | 1.9-2.1 | 8.2-8.4 |

Formula LR.17/9-A0.5, A1 and A1.5 were transparent, and formula LR.17/9-A2 was opaque.

### Example 7

The API % was tested between the levels for A1.5 and A2 in Example 6. The formulas tested were as follows: Lauroyl Macroglycerides (primary lipid) at 1-4%, Propylene Glycol Caprate (secondary lipid, high solubility of API) at 0.1-1%, PEG-35 Castor Oil (co-surfactant, anti-aggregation of nanoparticles) at 6-9%, API (Active Pharmaceutical Ingredient) at varying %, water (solution medium, NLC former) at 60-90%, Polysorbitan 20 (High HLB non-ionic surfactant, NLC former and stabilizer) at 6-9%. Table 8, below, shows the API ranges in the base formula LR.17/9 in sub-samples A1.6 and A1.8.

TABLE 8

| | API % w/w range in | API % range in |
|---|---|---|
| Code | Formula (Cannabidiol) | Nanoparticle |
| LR.17/9-A1.6 | 1.6-1.7 | 6.7-6.9 |
| LR.17/9-A1.8 | 1.8-1.9 | 7.4-7.6 |

Formulas were tested visually after preparation. Formula LR.17/9-A1.6 was transparent and formula LR.17/9-A1.8 was opaque. It appeared that 6.7-6.9 API % in nanostructured lipid carriers of Formula LR.17/9 was the maximum loading capacity.

### Example 8

Loading capacity was tested for the emulsifiers/surfactant by increasing the amount of lipids and API. Formulas for testing emulsifiers/surfactant capacity were as follows: Lauroyl Macroglycerides (primary lipid) at varying %, Propylene Glycol Caprate (secondary lipid, high solubility of API) at varying %, PEG-35 Castor Oil (co-surfactant, anti-aggregation of nanoparticles) at 6-9%, API (Active Pharmaceutical Ingredient) at varying %, water (solution medium, NLC former) at 60-90%, Polysorbitan 20 (High HLB non-ionic surfactant, NLC former and stabilizer) at 6-9%. Table 9, below, shows the formulas created and tested.

TABLE 9

| | LR.17/ | LR.17/ | LR.17/ |
|---|---|---|---|
| Formula Code | 9-B10 | 9-B25 | 9-B50 |
| Ingredients Under Analysis | % w/w | % w/w | % w/w |
| Lauroyl Macroglycerides | 4-5 | 5-6 | 6-7 |
| Glyceryl Caprylate | 1-1.2 | 1.25-1.35 | 1.5-1.6 |
| Cannabidiol | 1.6-1.8 | 2-2.2 | 2.4-2.5 |
| % API in Nanoparticle | 7.1-7.3 | 7.8-8.0 | 8.8-9.0 |
| (Cannabidiol) | | | |

Formulas were tested visually after preparation, where all formulas were opaque. It was decided to test an increased amount of lipids and API at and increase of 2.5%, 5% and 7.5%.

The references cited throughout this application, are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that the invention is not limited to the particular embodiments disclosed, but is intended to cover all modifications which are within the spirit and scope of the invention as defined by the appended claims, the above description, and/or shown in the attached drawings.

1. A composition comprising at least one nanostructured lipid carrier,
the nanostructured lipid carrier comprising a shell comprising an emulsifier, and an inner matrix comprising a solid lipid and/or a liquid lipid, wherein
the nanostructured lipid carrier has a diameter of 100 nm or less.

2.-6. (canceled)

7. The composition of claim 1, wherein the at least one nanostructured lipid carrier comprises a plurality of nanostructured lipid carriers, and the diameter is the average diameter of the plurality of nanostructured lipid carriers,
wherein the diameter is a value selected from a range having a low endpoint and a high endpoint, the low endpoint is selected from the group consisting of 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, and 49 nm, and the high endpoint is larger than the low endpoint and is selected from the group consisting of 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, and 50 nm.

8. The composition of claim 7 further comprising at least one active pharmaceutical ingredient in at least one of the plurality of nanostructured lipid carriers.

9. The composition of claim 8, wherein the at least one active pharmaceutical ingredient is hydrophobic.

10. The composition of claim 8, wherein the at least one active pharmaceutical ingredient is selected from the group consisting of a cannabinoid, cannabidiol, bicalutamide, carvediol, lovastatin, luteolin, mitotane, oridonin quercetin, spironolactone, saquinavir, saquinavir mesylate, testosterone undecanoate, thistle oil, safflower oil, sea buck thorn oil, carrot extract, thymoquinone, vinpocetine, and zerumbone.

11. The composition of claim 10, wherein the active pharmaceutical ingredient comprises cannabidiol.

12. The composition of claim 1, wherein the emulsifier is a PEG derivative.

13. The composition of claim 1, wherein the solid lipid is at least one selected from the group consisting of lauroyl macroglycerides, PEG-32 stearate, glyceryl dibehenate, hydrogenated coconut PEG-32 esters, glyceryl oleate, lauroyl macroglycerides, and tricaprin.

14. The composition of claim 1, wherein the liquid lipid is at least one selected from the group consisting of apricot kernel oil PEG-6 esters, corn oil PEG-6 ester, ethoxydiglycol, glyceryl distearate, polyglyceryl-3 dioleate, glyceryl linoleate, glyceryl oleate, glyceryl monooleate, propylene glycol monolaurate, PEG-8 caprylic/capric glycerides, polyglyceryl-3-diolate, propylene glycol caprate, propylene glycol laurate, PEG-8, caprylic/capric triglyceride, caprylic/capric triglyceride, PEG-8 carylic/capric glycerides, glyceryl caprylate/caprate, propylene glycol monocaprylate, Olea europaea (Olive) oil, and Glycine soja (Soyabean) oil.

15. The composition of claim 1, wherein the solid lipid comprises a lipid selected from glyceryl caprylate, glycerol dibehenate, and lauroyl macroglycerides, and the inner matrix comprises a liquid lipid selected from caprylic/capric triglyceride, glyceryl monooleate, propylene glycol monolaurate, hydrogenated coconut PEG-32 esters, glyceryl monooleate, and glyceryl caprylate.

16.-34. (canceled)

35. The composition of claim 11, wherein the cannabidiol is at a concentration of 7-15%.

36. (canceled)

37. The composition of claim 1 further comprising a soft gel, wherein the at least one nanostructured lipid carrier is incorporated in the soft gel.

38. A method of making a delivery system comprising nanostructured lipid carriers, the method comprising:
heating a water phase composition to at least a melting temperature;
heating an oil phase composition to at least the melting temperature;
mixing the water phase composition and the oil phase composition at least at the melting temperature to create a mixture; and
homogenizing the mixture in an aqueous medium to produce the nanostructured lipid carriers,
wherein
the water phase composition comprises an emulsifier and water,
the oil phase composition comprises a solid lipid and a liquid lipid, and the melting temperature is a temperature at which the solid lipid melts,
the water phase composition and the oil phase composition are liquid at or above the melting temperature, and
the solid lipid is solid below the melting temperature and the liquid lipid is liquid below the melting temperature, wherein
the homogenizing comprises drop-wise addition of the mixture into the aqueous medium and rapid agitation of the aqueous medium, and the aqueous medium is at an aqueous medium temperature lower than the melting temperature.

39.-42. (canceled)

43. The method of claim 38, wherein the aqueous medium temperature is 2-4° C. or 1-8° C., and the melting temperature is 200 above the solid lipid melting temperature, or 66° C., or 90° C.

44.-52. (canceled)

53. A method of making a delivery system comprising nanostructured lipid carriers, the method comprising:
at least one of filtering or drying the composition of claim 1 to prepare processed nanostructured lipid carriers.

54.-57. (canceled)

58. A method of treating a subject comprising administering the composition of claim 1 to a subject in need thereof.

59. The composition of claim 2 further comprising at least one active pharmaceutical ingredient in at least one of the plurality of nanostructure lipid carriers, at least one surfactant, and a solution medium.

60.-66. (canceled)

67. The composition of claim 59, wherein the at least one active pharmaceutical ingredient is hydrophobic.

68. The composition of claim 59, wherein the at least one active pharmaceutical ingredient is selected from the group consisting of a cannabinoid, cannabidiol, bicalutamide, carvediol, lovastatin, luteolin, mitotane, oridonin quercetin, spironolactone, saquinavir, saquinavir mesylate, testosterone undecanoate, thistle oil, safflower oil, sea buck thorn oil, carrot extract, thymoquinone, vinpocetine, and zerumbone.

69. The composition of claim 59, wherein the at least one active pharmaceutical ingredient comprises cannabidiol.

70.-89. (canceled)
